(19) United States
(12) Patent Application Publication
(10) Pub. No.: US 2011/0313052 A1
(43) Pub. Date: Dec. 22, 2011

(54) Composition Comprising Biodegradable Carrier for Controlled Delivery

(75) Inventor: Håkan Engqvist, Osthammar (SE)

(73) Assignee: ADURO MATERIAL AB, Osthammar (SE)

(21) Appl. No.: 13/148,686

(22) PCT Filed: Feb. 12, 2009
(86) PCT No.: PCT/SE2009/000080

§ 371 Date: Aug. 10, 2011

(51) Int. Cl. A61K 47/02; B02C 9/04

(52) U.S. Cl. 514/770; 241/25

(57) ABSTRACT

The present invention relates to a controlled release medical composition comprising: a powder composition of a binder; a water based liquid; and a medical active pharmaceutical ingredient. In a first embodiment the powder composition comprises at least calcium carbonate of a first phase and calcium carbonate of a second different phase, the first and second phase are selected from the group: amorphous calcium carbonate; vaterite; aragonite; and calcite; and in a second embodiment the powder composition comprises calcium carbonate, or calcium sulphate, or calcium phosphate, or combinations thereof.

### TECHNICAL FIELD

This application relates to a drug delivery system.

### BACKGROUND

API (active pharmaceutical ingredient) delivery is a term that refers to the delivery of a pharmaceutical compound to humans or animals. This is commonly also referred to as drug delivery. Most common methods of delivery include the preferred non-invasive oral (through the mouth), nasal, pneumonial (inhalation), and rectal routes. Many medications, however, can not be delivered using these routes because they might be susceptible to degradation or are not incorporated efficiently or they have a low solubility.

API candidates coming from combinatorial chemistry research and/or the API selected from biologically based high-throughput screening are quite often lipophilic. This challenges API delivery institutions in industry or academia to develop carrier systems for the optimal oral administration of these API. The increasing prevalence of poorly soluble API provides notable risk of the API showing low and erratic bioavailability particularly for API delivered by the oral route. To mention only a few of the challenges for this class of API: their oral bioavailability is poor and highly variable, including the massive use of surface-active excipients for solubilisation. Methods to solve these problems include; incorporation of the API into polymer carriers and the use of silica nanoparticles with the API attached to the particle. API in nanocrystalline form can be administered in smaller doses because they can be delivered directly to the tissue and in controlled doses. This increases the efficacy of the API. The API particle size is today mainly controlled via extensive milling processes or via the use of surface bonded API molecules on nanoparticles (silica). Also mesoporous silica has been proposed to be used as API delivery system.

Methods to solve the above mentioned problems for API and increase a API solubility and dissolution include; milling to reduce crystal (grain) size, crystallographic structure (i.e. crystalline or amorphous), crystal form and shape and the use of surfactants. Each these those methods have their drawbacks which might limit their applicability. An alternative approach available for the enhancement of API solubility, dissolution and bioavailability is through the application of co-crystals. This means that the physicochemical properties of the API and the bulk material properties can be modified, whilst maintaining the intrinsic activity of the API (N. Blagden et al, Advanced Drug Delivery Reviews 59 (2007) 617-630).

A range of drug delivery systems for local, controlled and/or targeted delivery has been developed in the past. Many are based on bioresorbable or biodegradable polymers, ceramics or hydrogels as carrier of API. Various calcium salt based ceramics, e.g. calcium phosphates have been described in the form of mouldable pastes to carry and release API, e.g. antibiotics (Royer U.S. Pat. Nos. 6,391,336, 6,630,486). Many of these systems have rapid delivery of the drug and/or a slow resorbtion rate, especially the calcium phosphate based systems.

Ceramic materials have been reported to be used as API carriers, e.g. silica and mesoporous silica have been proposed as API delivery materials for oral intake. Also calcium phosphate based materials and bioglasses have been proposed to be used as API carriers but not for oral intake (W. J. E. M. Habraken et al., Advanced Drug Delivery Reviews 59 (2007) 234-248) (Revisiting ceramics for medical applications, Maria Vallet-Reg, Dalton Trans., 2006, 5211-5220). Also their relatively high chemical stability makes them unsuitable to be used as solubility enhancers, the materials are proposed to be used for sustained release formulations. Calcium carbonate is also known to be used as excipient in tablet formulation and as Ca-source in tablets for delivery of Ca the bone.

New API delivery methods and API carrier materials that increases the API solubility, dissolution and bioavailability is highly needed. Also API delivery methods and API bioresorable carrier materials for targeted local delivery of API is highly needed.

### SUMMARY OF THE INVENTION

An object with the present invention is to provide an API carrier having an improved performance characteristics compared to prior art. Another object with the present invention is to provide a method to manufacture the carrier.

An advantage with the present invention is that the API carrier surprisingly has a controlled release function for the API. Another advantage with the present invention is that the API carrier surprisingly has an increased bioavailability/solubility function for the API.

Another advantage with the present invention is that it has a targeted local delivery function.

Further objects and advantages may be found by a skilled person in the art from the detailed description.

### DETAILED DESCRIPTION

A controlled release medical composition according to the invention may be designed to be delivered to treat or prevent a decease in to a subject in the need thereof either through oral delivery or local delivery, as described below. The medical composition includes an active pharmaceutical ingredient (API) that treats or prevents the decease.

Oral Delivery

A first embodiment of the present invention relates to a drug delivery system (DDS) for release of API's in order to be able to control the release and preferably increase the bioavailability/solubility of the API.

The DDS includes API combined with ceramic calcium compound based resorbable cement and optionally excipients and other methods to enhance solubility or controlled release, e.g. interior coating. A suitable calcium compound includes calcium carbonate, or calcium sulphate, or calcium phosphate, or combination thereof. The DDS refers to any method of administration preferably orally including capsule, powder, tablet, buccal or sublingual tablet, enetral, topical, inhalation.

Resorbable ceramics based on calcium phosphate or calcium sulphate as described in the literature, e.g. (Revisiting ceramics for medical applications, Maria Vallet-Reg, Dalton Trans., 2006, 5211-5220) and (WO 2005/039537) is often described to be used in sustained release formulations and often as injectable systems. Calcium carbonate is known to be used as excipient in tablet formulation and as Ca-source in tablets for delivery of Ca to the bone. In the unspecified form calcium carbonate as described above is not binding, i.e. can not be used as a cement that sets and harden. Calcium carbonate has also been proposed to be used as injectable cement for bone reconstruction (C. Combes et. Al, Biomaterials 27 (2006) 1945-1954).

The first embodiment of the invention relates to the use of calcium carbonate cement, or calcium sulphate cement, or calcium phosphate cement, or combinations thereof as a binder of API in it is structure. The main binding system is preferably composed of calcium carbonate with an optionally second binding system of maximum up to 49 wt. % composed of calcium sulphate or calcium phosphate or combinations of the two. In an alternative embodiment, the main binding system is composed of calcium sulphate of the α-phase. Preferably, the calcium phosphate cement comprises the following calcium compounds; anhydrous monocalcium phosphate, anhydrous dicalcium phosphate, dicalcium phosphate dihydrate, octacalcium phosphate, alfa-tricalcium phosphate, beta-tricalcium phosphate, amorphous calcium phosphate, calcium-deficient hydroxyapatite, non-stoichiometric hydroxlapatite, tetracalciumphosphate (TTCP) and combinations thereof.

The cement is formed via mixing of a powder composition of the binder, optionally with API added, and a water based liquid, optionally with an API dissolved in the liquid, to a paste, i.e. API is added in the powder composition, or in the liquid, or in both. The paste is let to harden into any given shape, e.g. blocks, granules or powder. Preferably the hardened cement (with API) is milled to a fine powder and mixed with common pharmaceutical ingredients according to the desired delivery form.

Preferably the API is dissolved in the water based liquid. To increase the solubility of the API the liquid can be heated or cooled compared to room temperature, preferably heated. Optionally the liquid can also have a reduced or increased pH. The water based liquid could also optionally have a reduced polarity via mixing with another liquid, e.g. ethanol or oil. Other means of increasing the solubility of a specific API in the liquid is also included in the invention. The API can be any type of API.

The powder is composed as a first binding system of combinations of amorphous calcium carbonate, vaterite, aragonite and calcite. Optionally a second binding system of up to 49 wt. % containing calcium phosphate (comprising the following calcium compounds; anhydrous monocalcium phosphate, anhydrous dicalcium phosphate, dicalcium phosphate dihydrate, octacalcium phosphate, alfa-tricalcium phosphate, beta-tricalcium phosphate, amorphous calcium phosphate, calcium-deficient hydroxyapatite, non-stoichiometric hydroxlapatite, tetracalciumphosphate (TTCP) and combinations thereof) and or calcium sulphate (mixtures alfa or beta structure, hydrated, non-hydrated, or hemihydrates) is combined with the first binding system. The first binding system can have any combination of the given calcium carbonate phases, preferably the following composition ranges (in wt. % of first binding system, sum total 100%) for the calcium carbonate phases:

| | |
|---|---|
| Amorphous calcium carbonate | 0-100 |
| Vaterite | 0-100 |
| Aragonite | 0-100 |
| Calcite | 0-70 |

Even More Preferred

| | |
|---|---|
| Amorphous calcium carbonate | 10-90 |
| Vaterite | 10-60 |
| Aragonite | 0-30 |
| Calcite | 0-30 |

Most Preferred

| | |
|---|---|
| Amorphous calcium carbonate | 30-90 |
| Vaterite | 10-30 |
| Aragonite | 0-10 |

During the hardening reaction the first binding system will change its initial composition to according to

Amorphous calcium carbonate→Amorphous calcium carbonate+Vaterite+Aragonite+Calcite (1)

Vaterite→Vaterite+Aragonite and Calcite (2)

Aragonite→Aragonite+Calcite (3)

Calcite does not form a binding system on its own but take part in the reaction as a nucleation site. The above mentioned reactions (1)-(3) can be complete or uncompleted meaning that in the hardened state there could be all phases present.

The manufacture of the calcium carbonate phases in described in (C. Combes et. Al, Biomaterials 27 (2006) 1945-1954). Note that all phases can be made as solid solutions with e.g. Magnesium and Strontium. Other variants are also applicable. The Vaterite can be manufactured according to the double decomposition method of calcium chloride solution and sodium carbonate solution at 30 degrees Celsius to give Vaterite. Aragonite can be manufactured according to the double decomposition method of calcium chloride solution and sodium carbonate solution at 100 degrees Celsius to give Aragonite. Amorphous calcium carbonate (ACC) prepared without solid solution has low stability and partly transform to vaterite, aragonite and calcite. More stable solid solution ACC can prepared as via decomposition of calcium chloride, magnesium chloride (and/or strontium chloride) solution and sodium hydrogencarbonate at ambient temperature. Solid solutions of vaterite, aragonite and calcite can also be manufactured according to the described decomposition of calcium chloride, magnesium chloride (and/or strontium chloride) but at elevated temperature. Solid solutions of the described calcium carbonate phases are here by also included in the invention.

The second binding system will form brushite, monetite or hydroxyapatite (HA) and calcium sulphate hydrate. The unhydrates phases may still be present in the hardened binding system, non-limiting examples comprising the following calcium compounds; anhydrous monocalcium phosphate, anhydrous dicalcium phosphate, dicalcium phosphate dihydrate, octacalcium phosphate, alfa-tricalcium phosphate, beta-tricalcium phosphate, amorphous calcium phosphate, calcium-deficient hydroxyapatite, non-stoichiometric hydroxlapatite, tetracalciumphosphate (TTCP) and combinations thereof and or calcium sulphate (mixtures alfa or beta structure, hydrated, non-hydrated, or hemihydrates) as studied using X-ray diffraction on the hardened carrier material.

The grain size of the binding system phases (first and second) is below 300 micrometer, preferably below 100 micrometer, even more preferred below 30 micrometer.

According to one embodiment of the invention, the powder mixture, and thus the finished DDS, can contain ballast material, which does not take part in the chemical reactions between the binding phase and the hydration liquid, but which is present as a solid phase in the finished DDS. According to one aspect of the invention, the powder mixture can therefore contain up to 50 percent by volume of ballast material. Non-limiting examples of ballast material can be milled DDS, calcite, HA, milled hardened cement and/or a resorbable polymer. Examples of resorbable polymers include but are not limited to: polylactic acids, polylactic-coglycolide-acids. The grain size of the ballast is below 1 mm, preferably below 100 micrometer.

The liquid or powder could optionally contain dispersion agents or gelating agents to control the reology or the amount of liquid in the calcium carbonate cement, the amounts is limited to 20 wt. % of the total weight of the powder and liquid combined. Non-limiting examples of dispersion agents includes polycarboxylic acids, cellulose, polyvinylalcohol, polyvinylpyrrolidone, starch, NTA, polyacrylic acids, PEG and combinations thereof.

The powder and liquid components described above are mixed to a paste. The liquid to powder ratio is between 0.2 to 20 (w/w). The paste is let to harden into any given shape, e.g. blocks, granules or powder. Preferably the hardened cement paste (NB with API included according to the method described above) is milled to a fine powder, preferable with a powder grain size of below 100 micrometer, even more preferred below 20 micrometer. Hardening can be performed at room temperature, at elevated temperature or at reduced temperature. The hardening can also be performed in gases, moist or in vacuum. Milling can optionally be performed using ball milling, planetary ball milling, jet milling or combinations thereof.

The formed powder, blocks (size below 1 mm) or granules (size below 1 mm) can optionally be mixed with common pharmaceutical excipients and API to any the above mentioned delivery forms. An excipient is an inactive substance used as a carrier for the active ingredients of a medication. Excipients are also sometimes used to bulk up formulations with very potent active ingredients, to allow for convenient and accurate dosage. In addition to their use in the single-dosage quantity, excipients can be used in the manufacturing process to aid in the handling of the active substance concerned.

The dosage of API in one oral delivery unit (e.g. one tablet) is below 5 grams preferably below 1 gram.

Surprisingly the formed powder has a better bioavailability than the API alone. Surprisingly the formed powder has a higher solubility and than the API alone. Surprisingly the formed powder allows very potent API's to be dosed with a high accuracy.

Local Delivery

A second embodiment of the present invention also relates to a drug delivery system (DDS) for targeted and/or sustained release of API's in order to be able to administering locally a controlled release pharmaceutical composition comprising one or more active substances.

Resorbable ceramics based on calcium phosphate or calcium sulphate as described in the literature, e.g. (Revisiting ceramics for medical applications, Maria Vallet-Reg, Dalton Trans., 2006, 5211-5220) and (WO 2005/039537) is often described to be used in sustained release formulations and often as injectable systems. Calcium carbonate is known to be used as excipient in tablet formulation and as Ca-source in tablets for delivery of Ca to the bone. In the unspecified form calcium carbonate as described above is not binding, i.e. can not be used as a cement that sets and harden. Calcium carbonate has also been proposed to be used as injectable cement for bone reconstruction (C. Combes et. al, Biomaterials 27 (2006) 1945-1954).

The second embodiment relates to the use of calcium carbonate cement as a binder of API in it is structure for local controlled release of the same. The main binding system is composed of calcium carbonate with an optionally second binding system of maximum up to 49 wt. % composed of calcium sulphate or calcium phosphate or combinations of the two. The cement is formed via mixing of a powder composition of the binder (optionally containing API) and a liquid (optionally with API dissolved in it) to a paste, i.e. API is added in the powder composition, or in the liquid, or in both. The paste solidifies after injection enveloping the API for a controlled targeted release of the same.

An API can optionally be dissolved in a water based liquid. To increase the solubility of the API the liquid can be heated or cooled compared to room temperature, preferably heated. Optionally the liquid can also have a reduced or increased pH. The water based liquid could also optionally have a reduced polarity via mixing with another liquid, e.g. ethanol. Other means of increasing the solubility of a specific API in the liquid is also included in the invention. The API can be any type of API.

The powder is composed as a first binding system of combinations of amorphous calcium carbonate, vaterite, aragonite and calcite. Optionally a second binding system of up to 49 wt. % containing calcium phosphate (mixtures of amorphous calcium phosphate, alfa-tricalciumphosphate, beta-tricalciumphosphate, MCPM) and or calcium sulphate (mixtures alfa or beta structure, hydrated, non-hydrated, or hemihydrates) is combined with the first binding system. The first binding system can have any combination of the given calcium carbonate phases, preferably the following composition ranges (in wt. % of first binding system, sum total 100%) for the calcium carbonate phases:

| | |
|---|---|
| Amorphous calcium carbonate | 0-100 |
| Vaterite | 0-100 |
| Aragonite | 0-100 |
| Calcite | 0-70 |

Even More Preferred

| | |
|---|---|
| Amorphous calcium carbonate | 10-90 |
| Vaterite | 10-60 |
| Aragonite | 0-30 |
| Calcite | 0-30 |

Most Preferred

| | |
|---|---|
| Amorphous calcium carbonate | 30-90 |
| Vaterite | 10-30 |
| Aragonite | 0-10 |

During the reaction the first binding system will change its initial composition to according to:

Amorphous calcium carbonate→Amorphous calcium carbonate+Vaterite+Aragonite+Calcite (1)

Vaterite→Vaterite+Aragonite and Calcite (2)

Aragonite→Aragonite+Calcite (3)

Calcite does not form a binding system on its own but take part in the reaction as a nucleation site. The above mentioned reactions (1)-(3) can be complete or uncompleted meaning that in the hardened state there could be all phases present.

The manufacture of the calcium carbonate phases in described in (C. Combes et. al, Biomaterials 27 (2006) 1945-1954). Note that all phases can be made as solid solutions with e.g. Magnesium and Strontium. Other variants are also applicable. The Vaterite can be manufactured according to the double decomposition method of calcium chloride solution and sodium carbonate solution at 30 degrees Celsius to give Vaterite. Aragonite can be manufactured according to the double decomposition method of calcium chloride solution and sodium carbonate solution at 100 degrees Celsius to give Aragonite. Amorphous calcium carbonate (ACC) prepared without solid solution has low stability and partly transform to vaterite, aragonite and calcite. More stable solid solution ACC can prepared as via decomposition of calcium chloride, magnesium chloride (and/or strontium chloride) solution and sodium hydrogencarbonate at ambient temperature. Solid solutions of vaterite, aragonite and calcite can also be manufactured according to the described decomposition of calcium chloride, magnesium chloride (and/or strontium chloride) but at elevated temperature. Solid solutions of the described calcium carbonate phases are here by also included in the invention.

The second binding system will form brushite or hydroxyapatite (HA) and calcium sulphate in addition to the added not reacted or partly reacted second binding phases.

The grain size of the binding system phases (first and second) is below 300 micrometer, preferably below 100 micrometer, even more preferred below 30 micrometer.

According to another embodiment of the invention, the powder mixture, and thus the finished DDS, can contain ballast material, which does not take part in the chemical reactions between the binding phase and the hydration liquid, but which is present as a solid phase in the finished DDS. According to one aspect of the invention, the powder mixture can therefore contain up to 50 percent by volume of ballast material. Non-limiting examples of ballast material can be milled DDS, calcite, HA, milled hardened cement and/or a resorbable polymer. Examples of resorbable polymers include but are not limited to: polylactic acids, polylactic-coglycolide-acids. The grain size of the ballast is below 1 mm, preferably below 100 micrometer.

According to another embodiment of the invention, however, the powder mixture can contain API.

The liquid or powder could optionally contain dispersion agents or gelating agents to control the reology or the amount of liquid in the calcium carbonate cement, the amounts is limited to 20 wt .% of the total weight of the powder and liquid combined. Non-limiting examples of dispersion agents includes polycarboxylic acids, cellulose, polyvinylalcohol, polyvinylpyrrolidone, starch, NTA, polyacrylic acids, PEG and combinations thereof.

The powder and liquid components described above are mixed to a paste. The liquid to powder ratio is between 0.2 to 20 (w/w), preferably between 0.3 and 3.

The paste can optionally be mixed with common pharmaceutical excipients and API to any the above mentioned delivery forms. An excipient is an inactive substance used as a carrier for the active ingredients of a medication. Excipients are also sometimes used to bulk up formulations with very potent active ingredients, to allow for convenient and accurate dosage. In addition to their use in the single-dosage quantity, excipients can be used in the manufacturing process to aid in the handling of the active substance concerned.

Due to the inherent properties of the ceramics contained in the composition, the composition is radio-opaque and observable with standard clinical radioscopy methods, thus the positioning of controlled release composition based on a biodegradable ceramic can easily be monitored during injection and during the treatment period by e.g. ultrasound imaging; magnetic resonance imaging; X-ray transmission imaging; computer tomography imaging; isotope based imaging including positron emission tomography or gamma camera/SPECT; magnetic- or radio-wave based positioning systems. Accordingly, it is possible to ensure that the controlled release composition predominantly reaches the targeted parts. In a preferred embodiment, the method of the invention includes such a monitoring. The radio-opaque properties of the controlled release composition can also be used to increase the accuracy of radiation treatment, thus providing the possibility of combining adjuvant/neo-adjuvant local hormone and anti-hormone treatment with high precision external beam radiotherapy with or without a brachy boost.

Monitoring with the methods mentioned above may also be employed during the treatment period. A preferred controlled release composition for use in a method according to the invention releases the active substance primarily by erosion and/or diffusion, i.e. in such a case, the degradation rate of the controlled release pharmaceutical composition is a means for in vivo monitoring the release rate of the one or more active substances. Normally, it is recommended that such a monitoring, if any, is done at predetermined intervals after the injection such as, e.g., about every 1 month, about every 2 months or about every 3 months after the first injection of the controlled release pharmaceutical composition into the prostate tissue.

As mentioned above, the controlled release pharmaceutical composition is visible in vivo in the subjects treated for monitoring and dose adjustment. Consequently, a dose of the controlled release composition may be corrected by an additional dose and the interindividual differences in degradation of the dosage form and release of the active substance may be monitored and accounted for with a higher precision rather than standardized protocol. Furthermore, during treatment the size of the prostate as well as the conditions within the prostate may change e.g. with respect to pH. Such changes may also give rise to correction of the dose or the required release of the active substance. In the event that the monitoring reveals a faster degradation than expected or it shows a significant degradation of the controlled release pharmaceutical composition, the subject treated will normally need an additional administration of one or more supplemental doses of the one or more active substances. This dose may be a burst/boost dose of the active substance and/or a further injection in the form of a controlled release pharmaceutical composition.

The controlled release pharmaceutical composition may be designed to release the active substance during a predetermined period of time. Normally, the release period is from about 1 week to about 6 months (such as, e.g., about 1 week, about 2 weeks, about 3 weeks, about 1 month, about 2 months, about 3 months and preferably about 6 months or longer after injection of the first injected controlled release pharmaceutical composition) and, accordingly, it may be necessary in any event to repeat administration of the controlled release composition at regular intervals (i.e. if the release period is about 1 month, renewed administration may take place from about 3 weeks to about 1 month after the first administration, whereas if the release period is about 6 months, renewed administration may take place from about 5 to about 6 months after the first administration). In some cases, it may also be necessary to supplement with a boost dose depending on the physician's diagnosis and choice of treatment.

Examples of treatments using the controlled release system include but are not limited to cancer treatment, vaccine and depot systems. Examples of API but not limited to; flutamide, 2-hydroxy-flutamide, bicalutamide. The dosage of flutamide, 2-hydroxy-flutamide, and bicalutamide is in the range of 0.1-1000 mg per day.

The amount of injected paste is below 10 ml, preferably below 5 ml.

Surprisingly the invention gives a resorbable targeted controlled release of API.

Active Pharmaceutical Ingredients

In the present context to the two embodiments, the term “API” is intended to denote a therapeutically, prophylactically and/or diagnostically active substance or a substance that has physiologic effect. The term is intended to include the API in any suitable form such as e.g. a pharmaceutically acceptable salt, complex, solvate or prodrug thereof of in any physical form such as, e.g., in the form of crystals, amorphous, crystalline, co-crystal or a polymorphous form or, if relevant, in any stereoisomer form including any enantiomeric or racemic form, or a combination of any of the above.

In a further embodiment according to the invention, the one or more active API is/are selected from the group comprising an androgen or a derivative thereof (including any salt form, any crystal form, any enantiomeric form), an anti-androgen or a derivative thereof, a nonsteroidal selective androgen receptor modulator or a derivative thereof, an oestrogen or a derivative thereof, an anti-oestrogen or a derivative thereof, a gestagen or a derivative thereof, an anti-gestagen or a derivative thereof, an oligonucleotide, a progestagen or a derivative thereof, a gonadotropin-releasing hormone or an analogue or derivative thereof, a gonadotropin inhibitor or a derivative thereof, a gonadotropin antagonists or a derivative thereof, an adrenal and/or prostate enzyme inhibitor, antibiotics, a cyclooxygenase inhibitor or a derivative thereof, an 5-alpha-reductase inhibitor, an alpha-adrenergic antagonist, a non-steroidal anti-inflammatory drug (NSAIDS), a corticosteroid, a HMG-CoA reductase inhibitor or a derivative thereof (statines), a membrane efflux and/or membrane transport protein, an immune system modulator, an angiogenesis inhibitor, and combinations thereof. The therapeutically, prophylactically and/or diagnostically active drug substance(s) may also be in the form of a pharmaceutically acceptable salt, the active enantiomeric form, solvate or complex thereof or in any suitable crystalline or amorphous form or it may be in the form of a prodrug. A combination of a non-steroidal antiandrogen, such as flutamide, 2-hydroxy-flutamide, bicalutamide, nilutamide or cyproterone acetate, megesterol acetate, together with 5-alpha reductase inhibitors, HMG-CoA reductase inhibitors (statines), cyclooxygenase inhibitors, non-steroidal anti-inflammatory drug (NSAIDS), corticosteroids, alphaadrenergic antagonists, estrogens, anti-cancer medicines (such as cyclophosphamide, 5-fluorouracil, vincristine, cisplatin, epirubicin, taxotere), radiation enhancement factors (hypoxic cytotoxins), or growth and anti-growth factors may further improve the therapeutic effect for any prostate related disease such as those mentioned herein.

Examples of treatments include but are not limited to neurological diseases, autoimmune and immunological diseases, infections, inflammations, metabolic diseases, obesitas, diseases in the uro-genital tract, cardiovascular diseases, hematopoietic, anticoagulant, thrombolytic and antiplatelet diseases, hypercholesterolemia, dyslipidemia, respiratory diseases, diseases of the kidney, gastrointestinal diseases, liver diseases, hormonal disruption, replacement, substitution and vitamins.

The invention is applicable to therapeutic agents in a broad sense, including androgens or derivates thereof (e.g. testosterone), antiandrogens (cyproteron, 10 flutamide, hydroxyflutamide, bicalutamide, nilutamide) or derivatives thereof, oestrogens or derivates thereof, anti-oestrogens (e.g. tamoxifen, toremifen) or derivates thereof, gestagens or derivates thereof, antigestagens or derivates thereof, oligonucleotides, progestagens or derivates thereof, gonadotropin-releasing hormone or analogues or derivates thereof, gonadotropin inhibitors or derivates thereof, adrenal 15 and prostate enzyme synthesis inhibitors (such as a-reductase inhibitors), membrane efflux and membrane transport proteins (such as PSC 833, verapamil), Such compounds include danazol, ketoconazole, mefenamic acid, nisoldipine, nifedipine, nicardipine, felodipine, atovaquone, griseofulvin, troglitazone glibenclamide and carbamazepine and other cytostatic agents, immune system modulators and angiogenesis inhibitors alone or in combinations. The invention also includes any other suitable pharmaceutical agents applied in soft tissues or organs for local or systemic sustained drug release.

Examples of active drug substances from various pharmacological classes for the use in the present clinical context include e.g. antibacterial agents, antihistamines and decongestants, anti-inflammatory agents, 35 antiparasitics, antivirals, local anaesthetics, antifungals, amoebicidals or trichomonocidal agents, analgesics, antianxiety agents, anticlotting agents, antiartritics, antiasthmatics, anticoagulants, anticonvulsants, antidepressants, antidiabetics, antiglaucome agents, antimalarials, antimicrobials, antineoplastics, antiobesity agents, antipsychotics, antihypertensives, auto-immune disorder agents, anti-impotence agents, anti-Parkinsonism agents, anti-Alzheimers agents, antipyretics, anticholinergics, anti-ulcer agents, blood-glucose-lowering agents, bronchodilators, central nervous system, cardiovascular agents, cognitive enhancers, contraceptives, cholesterol-reducing agents, agents against dyslipidermia, cytostatics, diuretics, germicidials, H-2 blockers, hormonal agents, anti-hormonical agents, hypnotic agents, inotropics, muscle relaxants, muscle contractants, physic energizers, sedatives, sympathomimetics, vasodilators, vasoconstrictors, tranquilizers, electrolyte supplements, vitamins, uricosurics, cardiac glycosides, membrane efflux inhibitors, membrane transport protein inhibitors, expectorants, purgatives, contrast materials, radiopharmaceuticals, imaging agents, peptides, enzymes, growth factors, vaccines, mineral trace elements.

### EXAMPLES

### Example 1

Amorphous calcium carbonate (ACC) and Vaterite and calcite powder of grain size below 30 micrometer where dry mixed in the relation 3:1 by weight (ACC:Vaterite). The Vaterite where manufactured according to the double decomposition method of calcium chloride solution and sodium carbonate solution at 30 degrees Celsius to give Vaterite. ACC was prepared using a mixture calcium chloride, magnesium chloride solution and sodium hydrogencarbonate at ambient temperature. The dry mixed powder where further mixed with bicalutamide in the relation 1:4 (bicalutamide: ceramic powder).

Water where separately mixed with cellulose (NTA 1 g/l).

The ceramic bicalutamide powder where mixed with the liquid in the relation liquid to powder of 1:2 to a paste. The paste where let to harden to a cylinder in a humid cabinet at 37 degrees Celsius. The drug release from the hardened cylinder where measured in vitro. The results showed a prolonged release of bicalutamide from the cylinder of over 24 hours.

### Example 2

Amorphous calcium carbonate (ACC) and Vaterite and calcite powder of grain size below 30 micrometer where dry mixed in the relation 3:1 by weight (ACC:Vaterite). The Vaterite where manufactured according to the double decomposition method of calcium chloride solution and sodium carbonate solution at 30 degrees Celsius to give Vaterite. ACC was prepared using a mixture calcium chloride, magnesium chloride solution and sodium hydrogencarbonate at ambient temperature.

Danazol was dissolved in water via heating to 50 degrees Celsius.

The ceramic powder where mixed with the warm liquid in the relation liquid to powder of 1:2 to a paste. The paste where let to harden to thin cake in a humid cabinet at 37 degrees Celsius. The cake where crushed and dry milled to a powder of grain size below 20 micrometer.

The release rate from the powder was compared to grains (same crystal size) of Danazol in pH2 in vitro. The release was faster from the ceramic/drug powder than from the API it self.

1. A controlled release medical composition comprising:)
1) a powder composition of a first binder comprising at least calcium carbonate of a first phase and calcium carbonate of a second different phase, said first and second phase are selected from the group: amorphous calcium carbonate; vaterite; aragonite; and calcite
2) a water based liquid; and
3) a medical active pharmaceutical ingredient.

2. The controlled release medical composition according to claim 1, wherein said powder composition further comprises a second binder comprising calcium sulphate, or calcium phosphate, or a combination thereof.

3. The controlled release medical composition according to claim 2, wherein second binder is up to 49 wt. % of said powder composition.

4. The controlled release medical composition according to claim 1, wherein said water based liquid is mixed with a polarity reducing liquid.

5. The controlled release medical composition according to claim 4, wherein the polarity reducing liquid is ethanol or oil.

6. A method of treating or preventing a disease comprising injecting a paste of said controlled release medical composition according to claim 1 in to a subject in the need thereof, wherein said medical active pharmaceutical ingredient treats or prevents said disease.

7. A controlled release medical composition comprising:
1) a powder composition comprising calcium carbonate, or calcium sulphate, or calcium phosphate, or combinations thereof;
2) a water based liquid; and
3) a medical active pharmaceutical ingredient.

8. The controlled release medical composition according to claim 7, wherein said powder composition comprises at least calcium carbonate of a first phase and calcium carbonate of a second different phase, said first and second phase are selected from the group: amorphous calcium carbonate; vaterite; aragonite; and calcite.

9. The controlled release medical composition according to claim 7, wherein said water based liquid is mixed with a polarity reducing liquid.

10. The controlled release medical composition according to claim 9, wherein the polarity reducing liquid is ethanol or oil.

11. A tablet produced by the process of:
1) hardening a paste of said medical composition according to claim 7, to form a hardened medical composition,
2) grinding said hardened medical composition, and
3) forming a tablet of the ground hardened medical composition.

12. The tablet according to claim 11, wherein the process further comprises mixing the hardened medical composition with pharmaceutical excipients and optionally API before grinding.

13. The tablet according to claim 12, wherein the process further comprises mixing the hardened medical composition with pharmaceutical excipients and API before grinding.

14. A method of treating or preventing a disease comprising administrating of a tablet according to claim 11 in to a subject in the need thereof, wherein said medical active pharmaceutical ingredient treats or prevents said disease.

15. The controlled release medical composition according to claim 2, wherein said water based liquid is mixed with a polarity reducing liquid.

16. The controlled release medical composition according to claim 3, wherein said water based liquid is mixed with a polarity reducing liquid.

17. The controlled release medical composition according to claim 8, wherein said water based liquid is mixed with a polarity reducing liquid.
